(19) United States
(12) Patent Application Publication
(10) Pub. No.: US 2020/0088823 A1
(43) Pub. Date: Mar. 19, 2020

(54) MRI METHOD FOR T1 MAPPING OF THE HEART USING A MAXIMUM LIKELIHOOD RECONSTRUCTION IN K-SPACE

(71) Applicant: KONINKLIJKE PHILIPS N.V., EINDHOVEN (NL)

(72) Inventors: CHRISTIAN STEHNING, HAMBURG (DE); PETER BOERNERT, HAMBURG (DE); THOMAS ERIK AMTHOR, HAMBURG (DE); MARIYA IVANOVA DONEVA, HAMBURG (DE); JOUKE SMINK, BEST (NL); MARC KOUWENHOVEN, BEST (NL)

(21) Appl. No.: 16/471,022

(22) PCT Filed: Dec. 20, 2017
(86) PCT No.: PCT/EP2017/083917

§ 371 Date: Jun. 19, 2019

(30) Foreign Application Priority Data
Dec. 22, 2016 (EP) 16206053.7

(51) Int. Cl. G01R 33/50; G01R 33/54; G01R 33/56; G01R 33/567

(52) U.S. Cl. CPC G01R 33/50; G01R 33/543; A61B 5/055; G01R 33/5673; G01R 33/5608

(57) ABSTRACT

The invention provides for a magnetic resonance imaging system (100) for acquiring magnetic resonance data (146) from a subject (118) from a region of interest (109) within an imaging zone (108). The magnetic resonance imaging system comprises a memory (134) for storing machine executable instructions (140) and pulse sequence commands (142). The pulse sequence commands are configured for controlling the magnetic resonance imaging system to perform magnetization preparation pulses which causes magnetization inversion within the region of interest and initiates a T1 relaxation process. The pulse sequence commands are configured for acquiring portions of the magnetic resonance data as discrete units during a rest and relaxation interval of a heart phase of the subject. The magnetic resonance imaging system further comprises a processor (130) for controlling the magnetic resonance imaging system. Execution of the machine executable instructions causes the processor to repeatedly: receive (202) an ECG signal (124) descriptive of the heart phase of the subject; detect (204) an onset of the rest and relaxation interval of the heart phase using the ECG signal; acquire (206) a portion (146) of the magnetic resonance data a predetermined delay after the onset of the rest and relaxation interval by controlling (200) the magnetic resonance imaging system with the pulse sequence commands, wherein the portion of the magnetic resonance data undersamples k-space; determine (208) an inversion delay (308, 502) for the portion of the magnetic resonance data using a timing of the magnetization preparation pulses and the onset of the rest and relaxation interval. Execution of the machine executable instructions further causes the processor to calculate a T1 map (150) of the region of interest using a maximum likelihood reconstruction that uses the magnetic resonance data and the inversion delay for each portion of the magnetic resonance data.

### FIELD OF THE INVENTION

The invention relates to magnetic resonance imaging, in particular to the acquisition of magnetic resonance data triggered by ECG signals for T1 mapping.

### BACKGROUND OF THE INVENTION

A large static magnetic field is used by Magnetic Resonance Imaging (MRI) scanners to align the nuclear spins of atoms as part of the procedure for producing images within the body of a patient. This large static magnetic field is referred to as the B0 field.

During an MRI scan, Radio Frequency (RF) pulses generated by one or more transmitter coils cause a so called B1 field. Additionally applied gradient fields and the B1 field cause perturbations to the effective local magnetic field. RF signals are then emitted by the nuclear spins and detected by one or more receiver coils. These RF signals are used to construct the MR images. These coils can also be referred to as antennas.

MRI scanners are able to construct images of either slices or volumes. A slice is a thin volume that is only one voxel thick. A voxel is a small volume element over which the MR signal is averaged, and represents the resolution of the MR image. A voxel may also be referred to as a pixel (picture element) herein if a single slice is considered.

Depending upon the acquisition method, various types of information can be mapped or imaged during magnetic resonance imaging. For example, a so called inversion recovery sequence is a type of T1 weighted imaging sequence. The acquisition of magnetic resonance data at multiple inversion times (TI) can be used to calculate the spatially dependent T1 value, which is commonly referred to as a T1 map.

A magnetic resonance imaging protocol that may be used to make T1 maps of the heart is referred to as the Modified Look-Locker Inversion Recovery (MOLLI) magnetic resonance imaging protocol. The MOLLI protocol is described in the journal article Messroghli et. al., “Modified Look-Locker Inversion Recovery (MOLLI) for High Resolution T1 Mapping of the Heart,” Magnetic Resonance in Medicine 52:141-146 (2004).

### SUMMARY OF THE INVENTION

The invention provides for a magnetic resonance imaging system, a computer program product, and a method in the independent claims.

Myocardial T1 maps are clinically useful. However, it can be difficult to make accurately T1 maps of the heart because of its rhythmic motion. Techniques that currently exist rely on for example an ECG signal to identify a rest and relaxation interval of the heart where imaging can be performed. To make a T1 map, for example a number of images embedded into an inversion recovery pulse sequence may be sampled at various inversion times. The intensity of images produced for various inversions times may be used to calculate a T1 map. The difficulty with this approach is that there is only a limited time in which the magnetic resonance data can be measured. As a practical matter, it is not possible to make multiple measurements at the same inversion time because there are small irregularities in the timing of the heart from beat to beat. The pulse sequence for acquiring the magnetic resonance data is started before the onset of the rest and relaxation interval of the heart is detected. This means that the exact inversion time that the magnetic resonance data is acquired at is not controllable.

Examples may provide for an improved means of making myocardial T1 maps by avoiding the reconstruction of intermediate images all together. Instead the magnetic resonance data acquired is reconstructed into a T1 map using a maximum likelihood reconstruction. The quality of the resulting T1 map is then increased as more magnetic resonance data at different inversion times is acquired. The application of maximum likelihood reconstructions to compressed sensing is discussed in Doneva et. al., “Compressed Sensing in Quantitative MRI.” MRI: Physics, Image Reconstruction, and Analysis. Eds. Angshul Majumdar, and Rabab Kreidieh Ward. CRC Press, 2015. 51-71. See Eq. 3.6 and section 3.3 of this reference in particular.

In one aspect, the invention provides for a magnetic resonance imaging system for acquiring magnetic resonance data from a subject for a region of interest within an imaging zone. The magnetic resonance imaging system comprises a memory for storing machine-executable instructions and pulse sequence commands. The pulse sequence commands are configured for controlling the magnetic resonance imaging system to perform magnetization preparation pulses, which cause magnetization inversion within a region of interest and initiates a T1 relaxation process. The pulse sequence commands are configured for acquiring portions of the magnetic resonance data as discrete units during a rest and relaxation interval of the subject's heart.

The magnetic resonance imaging system further comprises a processor configured for controlling the magnetic resonance imaging system. Execution of the machine-executable instructions cause the processor to repeatedly receive an ECG signal descriptive of a heart phase of the subject. The rest and relaxation interval is one phase of the heart phase of the subject. Execution of the machine-executable instructions further cause the processor to repeatedly detect an onset of a rest and relaxation of the heart phase using the ECG signal. Execution of the machine-executable instructions further cause the processor to repeatedly acquire a portion of the magnetic resonance data at a predetermined delay after the onset of the rest and relaxation interval by controlling the magnetic resonance imaging system with the pulse sequence commands.

The portion of the magnetic resonance data undersamples the k-space. By undersampling the k-space it is understood herein that the magnetic resonance data is not sampled sufficiently within one portion to reconstruct a magnetic resonance image that accurately detects the subject. Execution of the machine-executable instructions further cause the processor to repeatedly determine an inversion delay for the portion of the magnetic resonance data using a timing of the magnetization preparation pulse and the onset of the rest and relaxation interval. There are slight irregularities in the timing of the heart which make it difficult to trigger the acquisition of the magnetic resonance data at a precisely determined inversion delay. The portion of the magnetic resonance data is therefore labeled with the determined inversion delay. The magnetization preparation pulses are performed before the onset of the rest and relaxation interval. Using the timing of the pulse sequence commands the exact inversion delay for when the magnetic resonance data is acquired can be determined.

Execution of the machine-executable instructions further cause the processor to calculate a T1 map of the region of interest using a maximum likelihood reconstruction that uses the magnetic resonance data and the inversion delay for each portion of the magnetic resonance data. In maximum likelihood reconstructions, the k-space data is not reconstructed into an image before the T1 map is calculated. A model of the measured magnetic resonance data can be used to predict the measured magnetic resonance data. In maximum likelihood reconstructions, the most likely value of the T1 map is calculated using the measured magnetic resonance data directly. This is performed in k-space.

This magnetic resonance imaging system may have the advantage that the T1 maps that are calculated may be of higher resolution and quality than is possible with conventional T1 mapping magnetic resonance imaging techniques.

In another embodiment, the maximum likelihood reconstruction is formulated as an optimization problem. This embodiment may be advantageous because it may provide for a means of using magnetic resonance data which is not sampled sufficiently for reconstructing intermediate images in order to construct a conventional T1 map that looks at the T1 weighted images at discrete inversion delays.

In another embodiment, the optimization problem compares the magnetic resonance data to a data model. The data model is dependent on the T1 map and a spatially dependent spin density value. This embodiment may be beneficial because it provides an accurate model that may provide a high quality T1 map without the need to generate intermediate images.

In another embodiment, the data model is an approximation of spatially dependent longitudinal magnetization within the region of interest. The use of such a data model many be beneficial because the spatially dependent longitudinal magnetization is directly related to the intensity of T1 weighted images that use inversion recovery.

In another embodiment, the data model is further dependent upon the pulse sequence commands. This embodiment may be beneficial because it may help to take into account the specific timing and repetition of the pulse sequence commands. For example the data model may include additional parameters to account for signal effects unrelated to T1 relaxation, such as saturation effects introduced by the magnetic resonance measurement sequence. Such parameters may not necessarily be put directly into the model, but it may be possible to conclude from the fitted model what the influence of the magnetic resonance sequence or pulse sequence commands on the observed signal was. This may help to extract the exponential T1 signal.

In another embodiment, the comparison of the data model to the magnetic resonance data is performed for each inversion delay. The magnetic resonance data was acquired as discrete portions of magnetic resonance data and each portion of magnetic resonance data has a particular inversion delay. In this embodiment, the inversion delay for each portion of the magnetic resonance data is considered. This may be beneficial because it may provide for a highly accurate T1 map.

In another embodiment, execution of the machine-executable instructions further cause the processor to bin the magnetic resonance data into predetermined inversion delay bins using the inversion delay. The comparison of the data model to the magnetic resonance data is performed for each of the predetermined inversion delay bins. In this embodiment instead of using the inversion delay assigned to each portion of the magnetic resonance data the magnetic resonance data is first binned. This may be beneficial because it may reduce the computational complexity of the optimization problem. This may be beneficial in accelerating the calculation of the T1 map.

In another embodiment, the optimization problem compares the data model to the magnetic resonance data in k-space. This may be beneficial because it eliminates the need to sample k-space within one portion of the magnetic resonance data sufficient to generate an image. This enables more k-space data to be acquired which enables a more accurate T1 map to be reconstructed.

In another embodiment, the inversion recovery magnetic resonance imaging protocol is a modified look-locker inversion recovery magnetic resonance imaging protocol. This embodiment may be beneficial because the modified look-locker inversion recovery magnetic resonance imaging protocol is typically used for measuring T1 maps of the heart. Embodiments of the invention however modify this by performing the maximum likelihood reconstruction instead of producing images which are then used to fit the T1 values. This may provide for more accurate T1 maps of the heart.

In another embodiment, the magnetic resonance imaging system further comprises the ECG system for providing the ECG signal.

In another aspect, the invention provides for a computer program product comprising machine-executable instructions for execution by a processor controlling the magnetic resonance imaging system configured for acquiring magnetic resonance data from a subject from a region of interest within an imaging zone. Execution of the machine-executable instructions cause the processor to repeatedly receive an ECG signal descriptive of a heart phase of the subject. Execution of the machine-executable instructions further cause the processor to repeatedly detect an onset of a rest and relaxation of the heart phase using the ECG signal. Execution of the machine-executable instructions further cause the processor to repeatedly acquire a portion of the magnetic resonance data at a predetermined delay after the onset of the rest and relaxation interval by controlling the magnetic resonance imaging system with the pulse sequence commands.

The pulse sequence commands are configured for controlling the magnetic resonance imaging system to perform magnetization preparation pulses which causes magnetization inversion within a region of interest and initiates a T1 relaxation process. The pulse sequence commands are configured for acquiring portions of magnetic resonance data as discrete units during the rest and relaxation interval. The portion of the magnetic resonance data undersamples k-space. Execution of the machine-executable instructions further causes the processor to repeatedly determine an inversion delay for the portion of the magnetic resonance data using a timing of the magnetization preparation pulse and the onset of the rest and relaxation interval.

Execution of the machine-executable instructions further cause the processor to calculate a T1 map of the region of interest using a maximum likelihood reconstruction that uses the magnetic resonance data and the inversion delay for each portion of the magnetic resonance data.

In another embodiment, the maximum likelihood reconstruction is formulated as an optimization problem. The optimization problem compares the magnetic resonance data to a data model. The data model is dependent upon the T1 map and a spatially dependent spin density value. The data model is an approximation of a spatially dependent longitudinal magnetization within a region of interest.

In another aspect, the invention provides for a method of operating a magnetic resonance imaging system for acquiring magnetic resonance data from a subject from a region of interest within an imaging zone. The method comprises repeatedly receiving an ECG signal descriptive of a heart phase of the subject. The method further comprises repeatedly detecting an onset of a rest and relaxation interval of the heart phase using the ECG signal. The method further comprises repeatedly acquiring a portion of the magnetic resonance data at a predetermined delay after the onset of the rest and relaxation interval by controlling the magnetic resonance imaging system with pulse sequence commands. The pulse sequence commands are configured for controlling the magnetic resonance imaging system to perform magnetization preparation pulses which cause magnetization inversion within a region of interest and initiates a T1 relaxation process. The pulse sequence commands are configured for acquiring portions of the magnetic resonance data as discrete units during the rest and relaxation interval. The portion of the magnetic resonance data undersamples k-space. The method further comprises repeatedly determining an inversion delay for the portion of the magnetic resonance data using a timing of magnetization preparation pulses and the onset of the rest and relaxation interval.

The method further comprises calculating a T1 map of the region of interest using a maximum likelihood reconstruction that uses magnetic resonance data and the inversion delay for each portion of the magnetic resonance data.

In another embodiment, the method further comprises selecting the region of interest to include a heart of the subject.

In another embodiment, the maximum likelihood reconstruction is formulated as an optimization problem. The optimization problem compares the magnetic resonance data to a data model. The data model is dependent upon the T1 map and a spatially dependent spin density value. The data model is an approximation of spatially dependent longitudinal magnetization within the region of interest.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example a data may be retrieved over a modem, over the internet, or over a local area network. Computer executable code embodied on a computer readable medium may be transmitted using any appropriate medium, including but not limited to wireless, wire line, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. ‘Computer storage’ or ‘storage’ is a further example of a computer-readable storage medium. Computer storage may be any volatile or non-volatile computer-readable storage medium.

A ‘processor’ as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object oriented programming language such as Java, Smalltalk, C++ or the like and conventional procedural programming languages, such as the C programming language or similar programming languages and compiled into machine executable instructions. In some instances the computer executable code may be in the form of a high level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It is understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, pedals, wired glove, remote control, and accelerometer are all examples of user interface components which enable the receiving of information or data from an operator.

A ‘hardware interface’ as used herein encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, bluetooth connection, wireless local area network connection, TCP/IP connection, ethernet connection, control voltage interface, MIDI interface, analog input interface, and digital input interface.

A ‘display’ or ‘display device’ as used herein encompasses an output device or a user interface adapted for displaying images or data. A display may output visual, audio, and or tactile data. Examples of a display include, but are not limited to: a computer monitor, a television screen, a touch screen, tactile electronic display, Braille screen, Cathode ray tube (CRT), Storage tube, Bi-stable display, Electronic paper, Vector display, Flat panel display, Vacuum fluorescent display (VF), Light-emitting diode (LED) display, Electroluminescent display (ELD), Plasma display panel (PDP), Liquid crystal display (LCD), Organic light-emitting diode display (OLED), a projector, and Head-mounted display.

Magnetic Resonance (MR) data is defined herein as being the recorded measurements of radio frequency signals emitted by atomic spins using the antenna of a magnetic resonance apparatus during a magnetic resonance imaging scan. Magnetic resonance data is an example of medical imaging data. A Magnetic Resonance (MR) image is defined herein as being the reconstructed two or three dimensional visualization of anatomic data contained within the magnetic resonance imaging data.

It is understood that one or more of the aforementioned embodiments of the invention may be combined as long as the combined embodiments are not mutually exclusive.

### BRIEF DESCRIPTION OF THE DRAWINGS

In the following preferred embodiments of the invention will be described, by way of example only, and with reference to the drawings in which:

FIG. 1 illustrates an example of a magnetic resonance imaging system;

FIG. 2 shows a flow chart which illustrates a method of operating the magnetic resonance imaging system of FIG. 1;

FIG. 3 illustrates several different sampling techniques which can be used by magnetization preparation pulses for measuring data which can be used for reconstructing a T1 map;

FIG. 4 illustrates a method of assigning the inversion time to measured magnetic resonance data; and

FIG. 5 illustrates a further method of assigning the inversion time to measured magnetic resonance data.

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

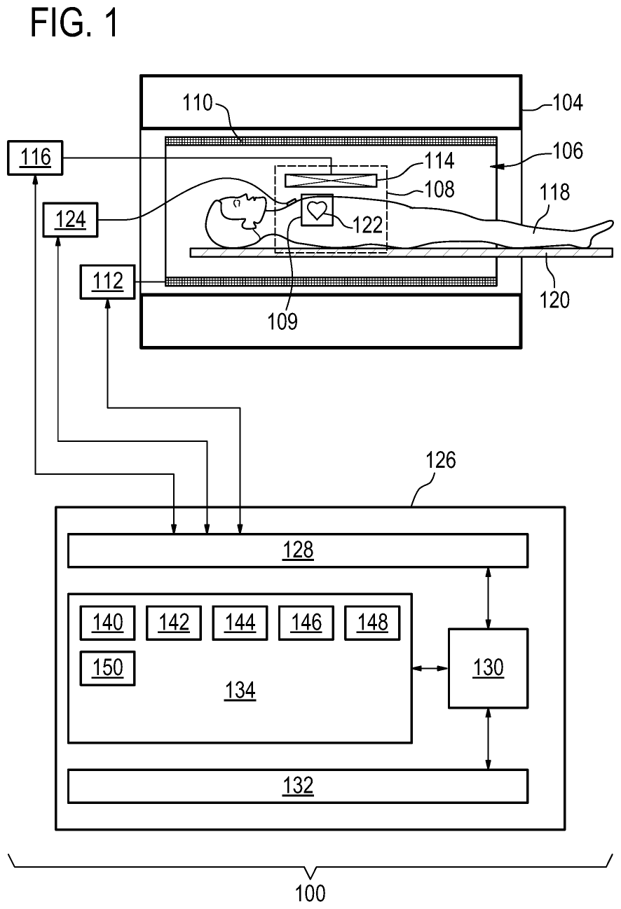

FIG. 1 shows an example of a magnetic resonance imaging system 100 with a magnet 104. The magnet 104 is a superconducting cylindrical type magnet with a bore 106 through it. The use of different types of magnets is also possible; for instance it is also possible to use both a split cylindrical magnet and a so called open magnet. A split cylindrical magnet is similar to a standard cylindrical magnet, except that the cryostat has been split into two sections to allow access to the iso-plane of the magnet, such magnets may for instance be used in conjunction with charged particle beam therapy. An open magnet has two magnet sections, one above the other with a space in-between that is large enough to receive a subject: the arrangement of the two sections area similar to that of a Helmholtz coil. Open magnets are popular, because the subject is less confined. Inside the cryostat of the cylindrical magnet there is a collection of superconducting coils. Within the bore 106 of the cylindrical magnet 104 there is an imaging zone 108 where the magnetic field is strong and uniform enough to perform magnetic resonance imaging. A region of interest 109 is shown within the imaging zone 108. A subject 118 is shown as being supported by a subject support 120 such that at least a portion of the subject 118 is within the imaging zone 108 and the region of interest 109. The heart 122 of the subject 118 can be seen as being within the region of interest 109. There is an ECG system 124 which connects one or more electrodes to the subject 118 to measure an ECG signal which is descriptive of a heart phase of the subject 118.

Within the bore 106 of the magnet there is also a set of magnetic field gradient coils 110 which is used for acquisition of magnetic resonance data to spatially encode magnetic spins within the imaging zone 108 of the magnet 104. The magnetic field gradient coils 110 connected to a magnetic field gradient coil power supply 112. The magnetic field gradient coils 110 are intended to be representative. Typically magnetic field gradient coils 110 contain three separate sets of coils for spatially encoding in three orthogonal spatial directions. A magnetic field gradient power supply supplies current to the magnetic field gradient coils. The current supplied to the magnetic field gradient coils 110 is controlled as a function of time and may be ramped or pulsed.

Adjacent to the imaging zone 108 is a radio-frequency coil 114 for manipulating the orientations of magnetic spins within the imaging zone 108 and for receiving radio transmissions from spins also within the imaging zone 108. The radio frequency antenna may contain multiple coil elements. The radio frequency antenna may also be referred to as a channel or antenna. The radio-frequency coil 114 is connected to a radio frequency transceiver 116. The radio-frequency coil 114 and radio frequency transceiver 116 may be replaced by separate transmit and receive coils and a separate transmitter and receiver. It is understood that the radio-frequency coil 114 and the radio frequency transceiver 116 are representative. The radio-frequency coil 114 is intended to also represent a dedicated transmit antenna and a dedicated receive antenna. Likewise the transceiver 116 may also represent a separate transmitter and receivers. The radio-frequency coil 114 may also have multiple receive/transmit elements and the radio frequency transceiver 116 may have multiple receive/transmit channels. For example if a parallel imaging technique such as SENSE is performed, the radio-frequency could 114 will have multiple coil elements.

In this example an ECG system 124 is shown as being a portion or part of the magnetic resonance imaging system 100. This is optional. In some examples the ECG system 124 may be a separate instrument and only the ECG signal 144 is provided.

The transceiver 116, the gradient controller 112, and the ECG system are shown as being connected to a hardware interface 128 of a computer system 126. The computer system further comprises a processor 130 that is in communication with the hardware system 128, a memory 134, and a user interface 132. The memory 134 may be any combination of memory which is accessible to the processor 130. This may include such things as main memory, cached memory, and also non-volatile memory such as flash RAM, hard drives, or other storage devices. In some examples the memory 130 may be considered to be a non-transitory computer-readable medium.

The computer memory 134 is shown as containing machine-executable instructions 140. The machine-executable instructions 140 contain commands which enable the processor 130 to control the operation and function of the magnetic resonance imaging system 100. The computer memory 134 is further shown as containing pulse sequence commands 142. The pulse sequence commands enable the processor 130 to acquire magnetic resonance data using the magnetic resonance imaging system 100. The computer memory 134 is further shown as containing an ECG signal 144 that was measured with the ECG system 124.

The computer memory 134 is further shown as containing a portion of the magnetic resonance data 146 acquired using the pulse sequence commands 142. The computer memory 134 is further shown as having data 148 that is descriptive of the inversion delay 146 of a particular portion of the magnetic resonance data 146. The computer memory 134 is further shown as containing a T1 map 150 that has been reconstructed using the multiple portions 146 of the magnetic resonance data.

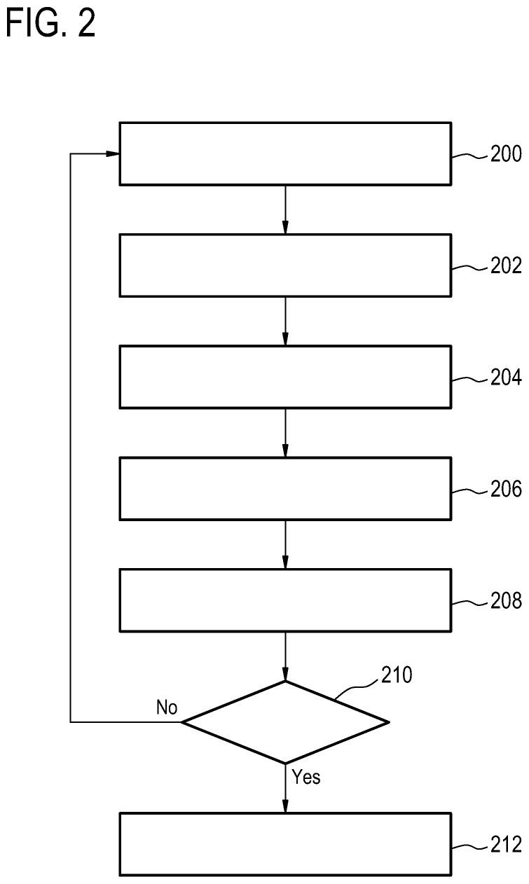

FIG. 2 shows a flowchart which illustrates a method of operating the magnetic resonance imaging system 100 of FIG. 1. First in step 200 the magnetic resonance imaging system 100 is controlled with the pulse sequence commands 142. The pulse sequence commands are configured for controlling the magnetic resonance imaging system to perform magnetization preparation pulses which cause magnetization inversion within the region of interest 109 and initiates a T1 relaxation process in the same region. The pulse sequence commands are configured for acquiring portions 146 of magnetic resonance data as discrete units during a rest and relaxation interval. Next in step 202 an ECG signal descriptive of a heart phase of the subject 118 is received. Next in step 204 an onset of the rest and relaxation interval of the heart phase is detected using the ECG signal 144. Then in step 206 the portion of the magnetic resonance data 146 is acquired a predetermined delay after the onset of the rest and relaxation interval.

Next in step 208 an inversion delay 148 is determined for the portion 146 of the magnetic resonance data. This is performed using a timing of the predetermined delay and the onset of the rest and relaxation interval. During steps 200-208 the portion of the magnetic resonance data has been acquired and the inversion delay for that portion has been determined. The method then proceeds to decision box 210. The question in this decision box is have all portions of the magnetic resonance data been acquired. If the answer is no the method returns back to step 200 and the next portion 146 of the magnetic resonance data is acquired. If all portions have been acquired the method then proceeds to step 212. In step 212 the T1 map 150 is calculated for the region of interest 109 using a maximum likelihood reconstruction that uses the magnetic resonance data 146 and the inversion delay 148 for each portion of the magnetic resonance data.

Examples may relate to quantitative MRI, namely cardiac T1 mapping, where a series of images are acquired at different inversion delays (TI) in order to obtain the T1 recovery time for each voxel by means of an exponential fit. The technique implies that images needs to be acquired in a “single shot” at specific TIs. The T1 map is then reconstructed by an exponential fit of a model to the reconstructed images.

Depending on the desired spatial resolution and coverage (=amount of data to be acquired for each image), it may be impossible to acquire all data in a single shot. The same restriction applies to an acquisition in subjects with high heart rates (Tachycardia patients, or small animals), in which the quiescent period of diastole is very short. Existing methods (e.g. SENSE) to reduce the amount of data needed to reconstruct an image with good spatial resolution are insufficient to overcome these limitations. Instead, it may be required to perform the acquisition of k-space data in a segmented fashion over a series of RR intervals instead. However, natural variations of the RR intervals results in data acquired partially at different inversion times (TI), which inhibits the reconstruction of images and subsequent fitting procedure.

Examples may provide for a segmented MR acquisition, where sufficient data to reach the desired spatial resolution and coverage are acquired in a segmented fashion over a series of RR intervals. An internal “bookkeeping” may be employed, where the inversion delay, i.e., the offset between the acquisition of the current k-space line and the last inversion pulse, is stored along with the data. The acquired data span a multidimensional matrix (e.g. a TI/ky-space in the case of a 2D acquisition). Unlike conventional mapping techniques, no images for specific inversion times, TI, will be reconstructed. Instead, a reconstruction algorithm such as a Maximum Likelihood Reconstruction is used to obtain the T1 value directly for each voxel from the available TI/ky data.

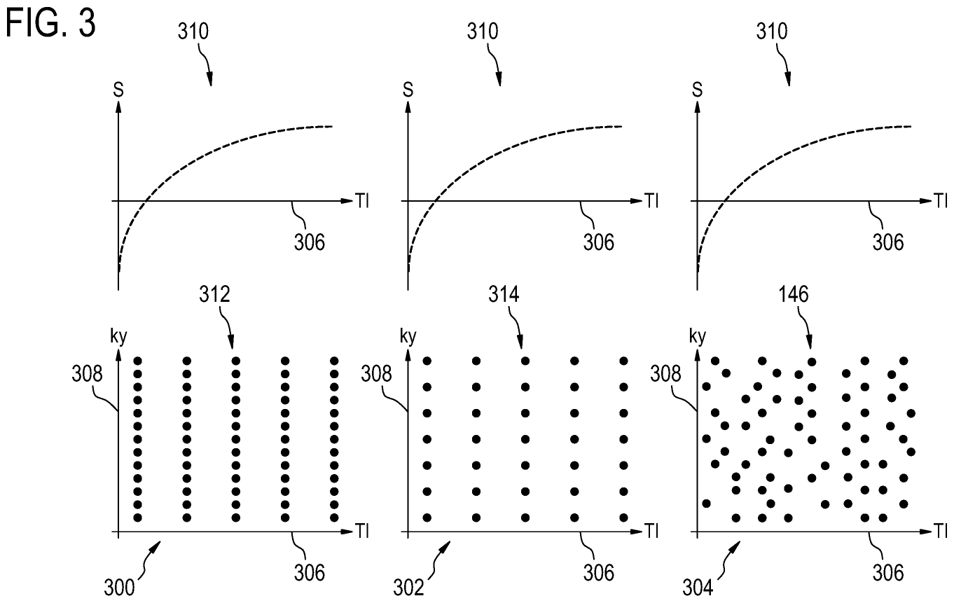

FIG. 3 illustrates several different sampling techniques which can be used by magnetization preparation pulses for measuring data which can be used for reconstructing a T1 map. The examples are divided into three columns. Column 300 represents the conventional k-space sampling scheme. Column 302 represents a SENSE k-space sampling scheme. Column 304 represents a maximum likelihood sampling scheme. Above each of the sampling schemes is a corresponding inversion recovery curve 310 which is included for illustrative purposes. It can be seen that the inversion recovery is identical in all three cases. The axis labeled 306 represents the inversion recovery or inversion delay. The axis 308 represents a location in k-space, in this case in the y-direction.

In the conventional 300 k-space sampling scheme there can be seen five different rows 312 which represent a portion of magnetic resonance data acquired after one magnetization preparation pulse. The data in each of these rows 312 is sufficient for reconstructing an image. The five images may then be reconstructed and may be used for fitting a T1 map.

Column 302 represents a SENSE reconstruction. The SENSE reconstruction enables the k-space data to be sampled more sparsely. There are again five rows 314 in k-space which are used in the SENSE sampling scheme. A fundamental limitation of the sampling schemes shown in rows 300 and 302 is that all of the data needs to be acquired in one row for a particular magnetization preparation pulse. The difficulty lies in that the data all needs to be acquired during the rest and relaxation interval of the subject's heart. As there are slight variabilities in this, the inversion delay may vary slightly during the various acquisitions.

Column 304 shows the maximum likelihood sampling scheme. One portion 146 of the magnetic resonance data is illustrated with the arrow 146. In the maximum likelihood reconstruction intermediate images are not reconstructed. It is therefore not necessary to acquire points in k-space that are sufficient to generate an image. It can also be seen that at different inversion delays the number of data points which are sampled is not even consistent. Because an intermediate image is not reconstructed there is no need for even the data within k-space to be sampled consistently or uniformly at different inversion delays. Any data that is acquired may be useful in contributing to and also to improving the resulting T1 map.

Eqs. (1) and (2) below are used to illustrate one numerical method of applying a maximum likelihood reconstruction. In T1 mapping, the magnetic resonance signal and inversion time TI can be modeled by an exponential recovery of the local magnetization:

X_{Ti}_{ⱼ}(ρ(\vec{r}),T1(\vec{r}))=ρ(\vec{r})−2p(\vec{r})e^{-TI}^{ⱼ}^{/T1(\vec{r})}, (1)

where row ρ(\vec{r}) is the local spin density,

where T1(\vec{r}) is the local tissue specific relaxation time, and

where TIⱼ is jth the inversion time. The index j can refer to the actual inversion time of groups of k-space points acquired together or they can refer to groups of k-space data points that are binned together and assigned an inversion time TIⱼ.

Equation (1) can be inserted into a optimization problem to solve it as a maximum likelihood reconstruction. An example of one formulation of such an optimization is:

$$( \frac{\hat{ρ}}{} ) = arg min \frac{1}{2} {\sum }_{j} {‖ f ( ρ ( \vec{r} ) - 2 p ( \vec{r} ) {e}^{- {TI}_{j} / T 1 ( \vec{r} )} ) - {y}_{{TI}_{j}} ‖}_{2}^{2} , \qquad ( 2 )$$

where

$$( \frac{\hat{ρ}}{} )$$

is spatially dependent local spin density map \hat{ρ} and T1 map [symbol] ,

where f represents taking a partial (or undersampled) Fourier transform, and

where y_{TI}_{ⱼ} is the measured Fourier data at the inversion time TIⱼ. The solution of optimization problems such as Eq. (2) are well known. In the formulation of Eq. (2) it is implicit that the measured Fourier data y_{TI}_{ⱼ} is calibrated. Even if the Fourier data y_{TI}_{ⱼ} is not calibrated, Eq. (2) still works, however the value of the spin density map \hat{ρ} will be scaled by a constant.

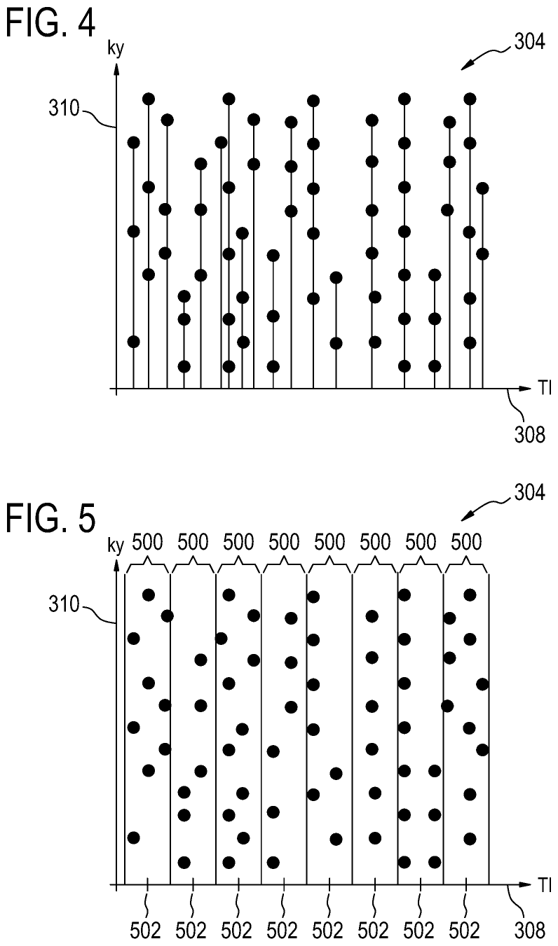

The measured magnetic resonance data y_{TI}_{ⱼ} can be used in equation 2 in several different ways. One way is to use the actual inversion time for each portion of the magnetic resonance data that is acquired. The portion of the magnetic resonance data acquired is represented in Eq. (2) as the y variable. FIGS. 4 and 5 are used to illustrate different ways of applying Eq. (2). In FIG. 4 another view of plot 304 from FIG. 3 is shown. There are vertical lines drawn through data points that correspond to the same inversion time 308. One means of applying Eq. (2) is to simply use data for every discrete single inversion time 308 indicated by a vertical line.

Another way of applying Eq. (2) is to first bin the magnetic resonance data. This may have the benefit of reducing the computational complexity of applying Eq (2). It can be seen that in FIG. 5 the inversion delay 308 has been divided into eight bins. Magnetic resonance data within each of these bins is then assigned to a particular inversion delay 502 for that bin 500. The scheme shown in FIG. 5 may in some cases produce a less accurate T1 map as using the scheme illustrated in FIG. 4, the scheme illustrated in FIG. 5 may have advantages in the computational efficiency and speed.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE NUMERALS

100 magnetic resonance imaging system
104 magnet
106 bore of magnet
108 imaging zone
109 region of interest
110 magnetic field gradient coils
112 magnetic field gradient coil power supply
114 radio-frequency coil
116 transceiver
118 subject
119 heart
120 subject support
122 heart
124 ECG system
126 computer system
128 hardware interface
130 processor
132 user interface
134 computer memory
140 machine executable instructions
142 pulse sequence commands
144 ECG signal
146 portion of the magnetic resonance data
148 inversion delay of 146
150 T1 map
200 control the magnetic resonance imaging system with the pulse sequence commands
202 receive an ECG signal descriptive of a heart phase of the subject
204 detect an onset of a rest and relaxation of the heart phase using the ECG signal
206 acquire a portion of the magnetic resonance data a predetermined delay after the onset of the rest and relaxation interval
208 determine an inversion delay for the portion of the magnetic resonance data a predetermined timing of one of the magnetization preparation pulses and the ECG signal
300 conventional k-space sampling scheme
302 SENSE k-space sampling scheme
304 maximum likelihood sampling scheme
306 iversion delay
308 location in k-space
310 corresponding inversion recovery curve
312 row in k-space conventional sampling
314 row in k-space SENSE sampling
500 predetermined inversion delay bins
502 inversion delay for bin

1. A magnetic resonance imaging system for acquiring magnetic resonance data from a subject from a region of interest within an imaging zone (108), wherein the magnetic resonance imaging system comprises:
a non-transitory computer readable memory for storing machine executable instructions and pulse sequence commands, wherein the pulse sequence commands are configured for controlling the magnetic resonance imaging system to perform magnetization preparation pulses which causes magnetization inversion within the region of interest and initiates a T1 relaxation process, wherein the pulse sequence commands are configured for acquiring portions of the magnetic resonance data as discrete units during a rest and relaxation interval of a heart phase of the subject;
a processor for controlling the magnetic resonance imaging system, wherein execution of the machine executable instructions causes the processor to repeatedly:
receive an ECG signal descriptive of the heart phase of the subject;
detect an onset of the rest and relaxation interval of the heart phase using the ECG signal;
acquire a portion of the magnetic resonance data a predetermined delay after the onset of the rest and relaxation interval by controlling (200) the magnetic resonance imaging system with the pulse sequence commands, wherein the portion of the magnetic resonance data undersamples k-space;
determine an inversion delay for the portion of the magnetic resonance data using a timing of the magnetization preparation pulses and the onset of the rest and relaxation interval; and
wherein execution of the machine executable instructions further causes the processor to calculate a T1 map of the region of interest using a maximum likelihood reconstruction that uses the magnetic resonance data and the inversion delay for each portion of the magnetic resonance data, wherein the magnetic resonance data within k-space are sampled inconsistently and/or non-uniformly at different inversion delays.

2. The magnetic resonance imaging system of claim 2, wherein the maximum likelihood reconstruction is formulated as an optimization problem.

3. The magnetic resonance imaging system of claim 2, wherein the optimization problem compares the magnetic resonance data to a data model, wherein the data model is dependent upon the T1 map and a spatially dependent spin density value.

4. The magnetic resonance imaging system of claim 3, wherein the data model is an approximation of a spatially dependent longitudinal magnetization within the region of interest.

5. The method of claim 3, wherein the data model is further dependent upon the pulse sequence commands.

6. The magnetic resonance imaging system of claim 3, wherein the comparison of the data model to the magnetic resonance data is performed for each inversion delay.

7. The magnetic resonance imaging system of claim 3, wherein execution of the machine executable instructions further cause the processor to bin the magnetic resonance data into predetermined inversion delay bins using the inversion delay, wherein the comparison of the data model to the magnetic resonance data is performed for each of the predetermined inversion delay bins.

8. The magnetic resonance imaging system of claim 3 wherein the optimization problem compares the data model to the magnetic resonance data in k-space.

9. The magnetic resonance imaging system of claim 1, wherein the inversion recovery magnetic resonance imaging protocol is a Modified Look-Locker Inversion Recovery magnetic resonance imaging protocol.

10. The magnetic resonance imaging system of claim 1, wherein the magnetic resonance imaging system further comprises an ECG system for providing the ECG signal.

11. A computer program product comprising machine executable instructions stored on a non-transitory computer readable medium for execution by a processor controlling a magnetic resonance imaging system configured for acquiring magnetic resonance data from a subject from a region of interest within an imaging zone, wherein execution of the machine executable instructions causes the processor to repeatedly:
receive an ECG signal descriptive of a heart phase of the subject;
detect an onset of a rest and relaxation interval of the heart phase using the ECG signal;
acquire a portion of the magnetic resonance data a predetermined delay after the onset of the rest and relaxation interval by controlling the magnetic resonance imaging system with pulse sequence commands, wherein the pulse sequence commands are configured for controlling the magnetic resonance imaging system to perform magnetization preparation pulses which causes magnetization inversion within a region of interest and initiates a T1 relaxation process, wherein the pulse sequence commands are configured for acquiring the portion of the magnetic resonance data a a discrete units during the rest and relaxation interval, wherein the portion of the magnetic resonance data undersamples k-space;
determine an inversion delay for the portion of the magnetic resonance data using a timing of the magnetization preparation pulses and the onset of the rest and relaxation interval; and
wherein execution of the machine executable instructions further causes the processor to calculate a T1 map of the region of interest using a maximum likelihood reconstruction that uses the magnetic resonance data and the inversion delay for each portion of the magnetic resonance data, wherein the magnetic resonance data within k-space are sampled inconsistently and/or non-uniformly at different inversion delays.

12. The computer program product of claim 11, wherein the maximum likelihood reconstruction is formulated as an optimization problem, wherein the optimization problem compares the magnetic resonance data to a data model, wherein the data model is dependent upon the T1 map and a spatially dependent spin density value, and wherein the data model is an approximation of spatially dependent longitudinal magnetization within the region of interest.

13. A method of operating a magnetic resonance imaging system for acquiring magnetic resonance data from a subject from a region of interest within an imaging zone,
wherein the method comprises repeatedly:
receiving an ECG signal descriptive of a heart phase of the subject;
detecting an onset of a rest and relaxation interval of the heart phase using the ECG signal;
acquiring a portion of the magnetic resonance data a predetermined delay after the onset of the rest and relaxation interval by controlling (200) the magnetic resonance imaging system with pulse sequence commands, wherein the pulse sequence commands are configured for controlling the magnetic resonance imaging system to perform magnetization preparation pulses which causes magnetization inversion within a region of interest and initiates a T1 relaxation process, wherein the pulse sequence commands are configured for acquiring the portion of the magnetic resonance data as a discrete unit during the rest and relaxation interval, wherein the portion of the magnetic resonance data undersamples k-space;
determine an inversion delay for the portion of the magnetic resonance data using a timing of the magnetization preparation pulses and the onset of the rest and relaxation interval; and
wherein the method further comprises calculating a T1 map of the region of interest using a maximum likelihood reconstruction that uses the magnetic resonance data and the inversion delay for each portion of the magnetic resonance data, wherein the magnetic resonance data within k-space are sampled inconsistently and/or non-uniformly at different inversion delays.

14. The method of claim 13, wherein the method further comprises selecting the region of interest to include a heart of the subject.

15. The method of claim 13 wherein the maximum likelihood reconstruction is formulated as an optimization problem, wherein the optimization problem compares the magnetic resonance data to a data model, wherein the data model is dependent upon the T1 map and a spatially dependent spin density value, and wherein the data model is an approximation of spatially dependent longitudinal magnetization within the region of interest.
